(19) United States
(12) Patent Application Publication
(10) Pub. No.: US 2018/0154029 A1
(43) Pub. Date: Jun. 7, 2018

(54) STERILIZING DEVICE AND MANUFACTURING METHOD FOR STERILIZING DEVICE

(71) Applicant: Industrial Technology Research Institute, Hsinchu (TW)

(72) Inventors: Ren-Chin Shr, Hsinchu City (TW); Teng-Chun Wu, Kinmen County (TW); Wei-Yun Liang, Hsinchu City (TW); Chih-Wei Kuo, Tainan City (TW)

(73) Assignee: Industrial Technology Research Institute, Hsinchu (TW)

(21) Appl. No.: 15/886,850

(22) Filed: Feb. 2, 2018

Related U.S. Application Data
(63) Continuation of application No. 13/050,501, filed on Mar. 17, 2011.
(60) Provisional application No. 61/347,933, filed on May 25, 2010.

(51) Int. Cl. A61L 2/10; A61N 5/06

(52) U.S. Cl. CPC A61L 2/10; A61N 5/0624; Y10T 29/49002

(57) ABSTRACT

A sterilizing device comprises a light guiding member and an ultraviolet (UV) light source. The light guiding member has a surface. The UV light source emits UV light rays such that the UV light rays are guided into the guiding member based on a total internal reflection. When an object contacts or comes close to the surface, an evanescent wave from the UV light rays irradiates on the object.

### 1. CROSS-REFERENCE TO RELATED APPLICATIONS

This application is a continuation application of and claims the priority benefit of a prior application Ser. No. 13/050,501, filed on Mar. 17, 2011, now pending. The prior application Ser. No. 13/050,501 is an application under 35 USC 111(a) and claims priority under 35 USC 119 from Provisional Application Ser. No. 61/347,933, filed May 25, 2010 under 35 USC 111(b). The entirety of each of the above-mentioned patent applications is hereby incorporated by reference herein and made a part of this specification.

### 2. TECHNICAL FIELD

The present disclosure relates to a sterilizing device and a manufacturing method for a sterilizing device.

### 3. BACKGROUND

Virus and bacteria are easily introduced into a human body through the subject's hands when the subject operates public facilities by physically touching a surface of a touch activation device such as a touch switch. Examples of such public facilities include elevators, information terminals, security panels, touch panels, automatic teller machines, etc. For example, the virus and bacteria may be present on elevator buttons after being contacted by a person with an infectious disease, and the pathogens could be spread when other people touch the same button.

A variety of photocatalyst devices have been disclosed to eliminate infectious germs from device surfaces, and thus prevent spread of infection. For example, an issued patent disclosed a photocatalytic glass pane equipped with a light source for photochemically activating or exciting a photocatalytic film on the glass pane, another issued patent disclosed a device and a reactor including a photocatalyst, and the other issued patent disclosed photocatalyst excitation apparatuses. However, these patents devices all require a photocatalyst which has the disadvantage of long reaction time and which is easily consumed on the surface of the object.

A published patent disclosed another structure using UV transmitting material and UV scattering material to introduce UV sterilizing radiation into an object to be sterilized. However, high intensity of UV radiation dose is harmful to human eyes and skin. Therefore, to reduce such danger, the patent employs relatively low intensity UV radiation for sterilization. The sterilizing process may require several hours or several days to kill the microorganisms on the surface, and thus the sterilizing efficiency is poor. Another operation mode of the patent is to increase the intensity of the UV radiation to improve the sterilizing efficiency when humans are not exposed to the UV light source. The foregoing conditions limit the applications of the patent.

Accordingly, there is a need to provide a sterilizing device for a touch activation device so as to disinfect a contact area when a user physically contacts or comes close to the contact area of the touch activation device. Another object of the present disclosure is to provide a germ-free surface of a sterilizing device. The germ-free surface is implemented by a predetermined time interval rather than by touch, and UV light rays within a light guiding member could not irradiate outside the sterilizing device during the sterilizing process. The light guiding member could be composed of a substantially transparent material, and thus is suitable for applications such as touch panels.

### SUMMARY

According to one embodiment of the present disclosure, the sterilizing device comprises a light guiding member and an ultraviolet (UV) light source. The light guiding member has a surface. The UV light source emits UV light rays such that the UV light rays are guided into the guiding member based on a total internal reflection. When an object contacts or comes close to the surface, an evanescent wave from the UV light rays irradiates on the object.

According to another embodiment of the present disclosure, the sterilizing device comprises a light guiding member and an ultraviolet (UV) light source. The light guiding member has a surface. The UV light source emits UV light rays such that the UV light rays are guided into the guiding member. When an object contacts or comes close to the surface, the UV light rays irradiate on the object due to a frustrated total internal reflection phenomenon.

Embodiment of the present disclosure is to provide a manufacturing method for a sterilizing device. According to one embodiment of the present disclosure, the method comprises the step of providing the sterilizing device, including the light guiding member having a surface, and an ultraviolet (UV) light source emitting UV light rays so that the UV light rays are guided into the light guiding member based on a total internal reflection. When an object contacts or comes close to the surface, an evanescent wave from the UV light rays irradiates on the object.

According to one embodiment of the present disclosure, the sterilizing touch panel comprises a display layer, a transparent touch screen, a light guiding member, a spacer, and an ultraviolet (UV) light source. The transparent touch screen is formed on the display layer. The light guiding member has a surface. The spacer is disposed between the transparent touch screen and the light guiding member. The UV light source emits UV light rays such that the UV light rays are guided into the guiding member based on a total internal reflection. When an object contacts or comes close to the surface, the UV light rays irradiate on the object due to a frustrated total internal reflection phenomenon.

The sterilizing device of the disclosure could be used in a variety of applications, for example, a publicly accessible apparatus having a manual activation device. According to one embodiment, the sterilizing device could be implemented as a touch panel, a door handle, an automatic door switch, and a touch mobile phone. During operation, when a user physically touches the front surface of the light guiding member of the sterilizing device, an evanescent wave goes out of the front surface and then propagates along the surface of the light guiding member. Therefore the contact area of the user will be disinfected by UV light rays. The sterilizing device could also sterilize the surface, if there are pathogens adhere to the surface, the evanescent UV light rays will irradiate on them and kill the pathogens on the surface.

The foregoing has outlined rather broadly the features and technical advantages of the disclosure in order that the detailed description of the disclosure that follows may be better understood. Additional features and advantages of the disclosure will be described hereinafter, and form the subject of the claims of the disclosure. It should be appreciated by those skilled in the art that the conception and specific embodiment disclosed might be readily utilized as a basis for modifying or designing other structures or processes for carrying out the same purposes of the disclosure. It should also be realized by those skilled in the art that such equivalent constructions do not depart from the spirit and scope of the disclosure as set forth in the appended claims.

### BRIEF DESCRIPTION OF THE DRAWINGS

The accompanying drawings, which are incorporated in and constitute a part of this specification, illustrate embodiments of the disclosure and, together with the description, serve to explain the principles of the disclosure.

FIG. 1 shows a cross-sectional view of a sterilizing device 10 in accordance with an exemplary embodiment;

FIG. 2 shows a cross-sectional view of a sterilizing device in accordance with an exemplary embodiment;

FIG. 3A is an illustration of a cross-sectional view showing the formation of guided light rays;

FIG. 3B is an illustration of a cross-sectional view showing the formation of unguided light rays;

FIG. 4 provides a visual explanation of an evanescent wave. The figure is an example of the field distribution for Transverse-Electric (TE) guided modes in the dielectric light guiding slab;

FIGS. 5A and 5B show a cross-sectional view of a sterilizing switch button device 50 in accordance with an exemplary embodiment;

FIG. 6 shows the flow chart of one embodiment of a sterilizing method of the present disclosure;

FIG. 7 shows the flow chart of another embodiment of a sterilizing method of the present disclosure;

FIG. 8 shows a cross-sectional view of a sterilizing touch panel in accordance with an exemplary embodiment;

FIG. 9A shows a sterilizing device in accordance with an exemplary embodiment;

FIG. 9B shows one embodiment of the sterilizing device of FIG. 9A with more detail;

FIG. 9C shows another embodiment of the sterilizing device of FIG. 9A with more detail;

FIG. 10 shows another arrangement of a UV light source in a sterilizing device in accordance with an exemplary embodiment;

FIG. 11 shows another arrangement of a UV light source in a sterilizing device in accordance with an exemplary embodiment;

FIG. 12 shows another arrangement of a UV light source in a sterilizing device in accordance with an exemplary embodiment;

FIG. 13 shows another arrangement of a UV light source in a sterilizing device in accordance with an exemplary embodiment;

FIG. 14 shows another arrangement of a UV light source in a sterilizing device in accordance with an exemplary embodiment; and

FIG. 15 shows another arrangement of a UV light source in a sterilizing device in accordance with an exemplary embodiment.

### DETAILED DESCRIPTION

Exemplary embodiments will now be described more fully with reference to the accompanying drawings. The embodiments may, however, be embodied in many different forms and should not be construed as limited to the embodiments set forth herein. Rather, these embodiments are provided so that this disclosure will be thorough and complete, and will fully convey the scope of the embodiments to those skilled in the art.

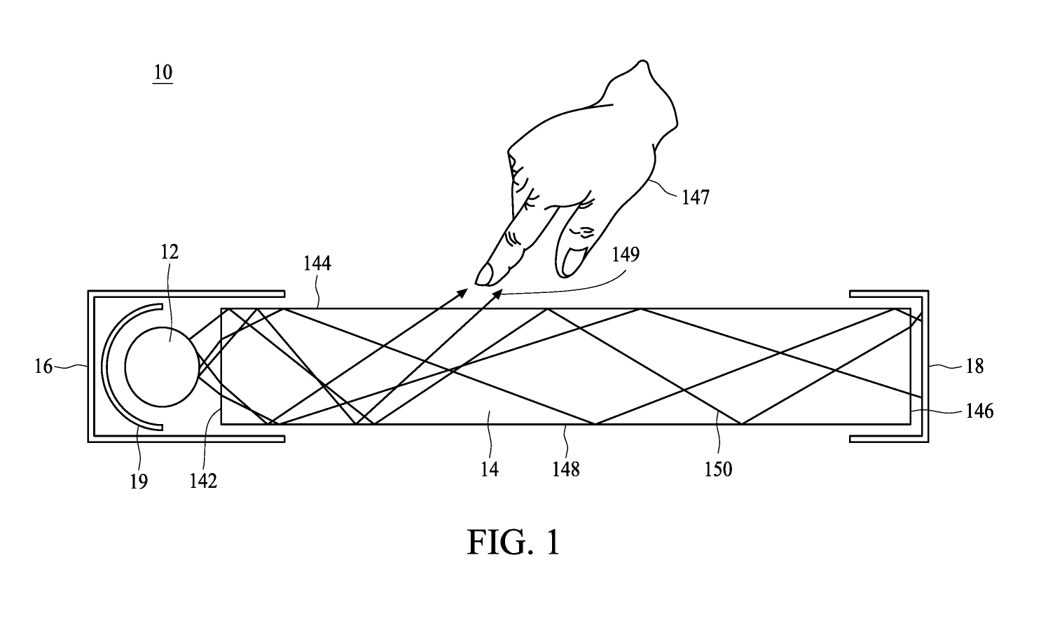

FIG. 1 shows a cross-sectional view of a sterilizing device 10 in accordance with an exemplary embodiment. The sterilizing device 10 comprises a short wavelength light source 12 and a slab of dielectric material as a light guiding member 14. In this embodiment, the light source 12 is an ultraviolet (UV) light source configured to generate ultraviolet light rays (a ray is an idealized narrow beam of light) or an ultraviolet light beam for sterilization. Generally, UV light rays are classified into four types: UV-A light rays having wavelength from 320 nm to 400 nm, UV-B light rays having wavelength from 280 nm to 320 nm, UV-C light rays having wavelength from 190 nm to 280 mm, and Vacuum UV (VUV) light rays having wavelength shorter than 190 nm. All kinds of these UV light rays could kill pathogens, but UV-C light rays are most efficient for killing pathogens.

The light source 12 may be made from florescent lamp, Cold Cathode Fluorescent Lamp (CCFL), Light-emitting diode (LED), deuterium lamp, gas discharge lamp, metal-vapour discharge lamps, xenon lamp, etc.

In one embodiment of the present disclosure, the light guiding member 14 may be made from inorganic material such as glass, borosilicate glass, fused silica, quartz, sapphire, LiF, MgF₂, CaF₂, BaF₂, plastic, or polymers (e.g. Teflon FEP), etc., or it may be made of organic material such as silicone resin such as dimethyl silicone, acrylic resin such as methacrylate, polyethylene, polycarbonate resin, or UV transmissible fluoric resin such as polyfluoroethylene, etc. In another embodiment of the present disclosure, the light guiding member 14 may be made from plastic, and thus the light guiding member is flexible.

Referring to FIG. 1, the light guiding member 14 has side surfaces 142 and 146, a front surface 144, and a rear surface 148. The front surface 144 and rear surface 148 is smooth so as to prevent scattering of the UV light. The light source 12 could be composed of a lamp with a tubular shape and is disposed adjacent to the side surface 142 of the light guiding member 14. As shown in FIG. 1, the light source 12 and parts of the front surface 144 and rear surface 148 adjacent to the light source 12 are covered by a covering member 16, and the side surface 146 and parts of the front surface 144 and rear surface 148 of the light guiding member 14 are covered by a covering member 18. In this manner the light rays which could not be guided in the light guiding member 14 are absorbed by the covering members 16 and 18, so that a user close to the sterilizing device 10 is not exposed to the light rays from the edge of the light guiding member 14. In addition, a reflector 19 is disposed adjacent to the light source 12 to enhance the coupling efficiency of the light source 12, and the intensity of the guided light rays could be increased in this manner.

Referring to FIG. 1, some ultraviolet light rays radiating from the ultraviolet light source 12 are introduced into the side surface 142 and coupled into the light guiding member 14, and then the ultraviolet light rays are guided within the light guiding member 14 due to the Total Internal Reflection (TIF) effect. Therefore, the guided light rays 150 could not leak out of the front surface 144 and the rear surface 148. In addition, when an object, for example, a human finger, contacts or comes close to the front surface 144 of the light guiding member 14 as shown in FIG. 1, some guided light rays 150 will penetrate through the interface and irradiate on the area of the finger skin near to the interface. As shown in FIG. 1, light rays 149 penetrate through the front surface 144 and irradiate at the contact area of the human finger 147. This phenomenon is known as a Frustrated Total Internal Reflection (FTIR) phenomenon or an evanescent wave phenomenon. Typically, when there is a total refection, an evanescent wave is formed at the boundary. The evanescent wave exhibits rapid exponential decay away from the boundary, so that it acts only on objects very close to the boundary, with the effective distance being several micrometers, depend on the wavelength. Because the evanescent wave only affects objects very close to the boundary, the device is very safe for using in daily life even if there are high intensity UV light rays inside the light guide.

In addition, the present disclosure is to provide a manufacturing method for a sterilizing device 10. According to one embodiment of the present disclosure, the method comprises the step of providing the sterilizing device 10, including the light guiding member 14 having a front surface 144, and an ultraviolet light source 12 emitting UV light rays so that the some light rays are guided into the light guiding member 14 based on a total internal reflection. When an object contacts or comes close to the surface, an evanescent wave from the UV light rays irradiates on the object.

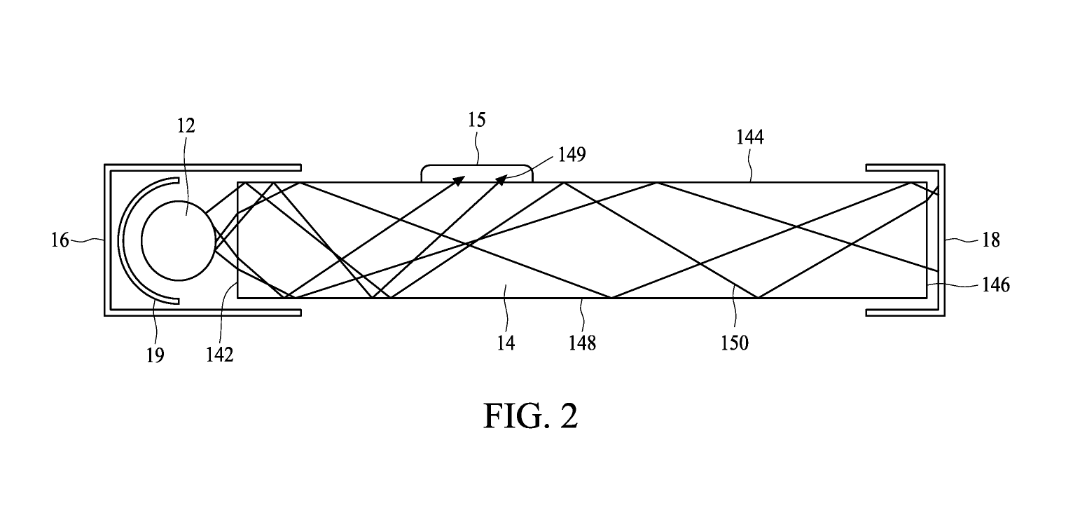

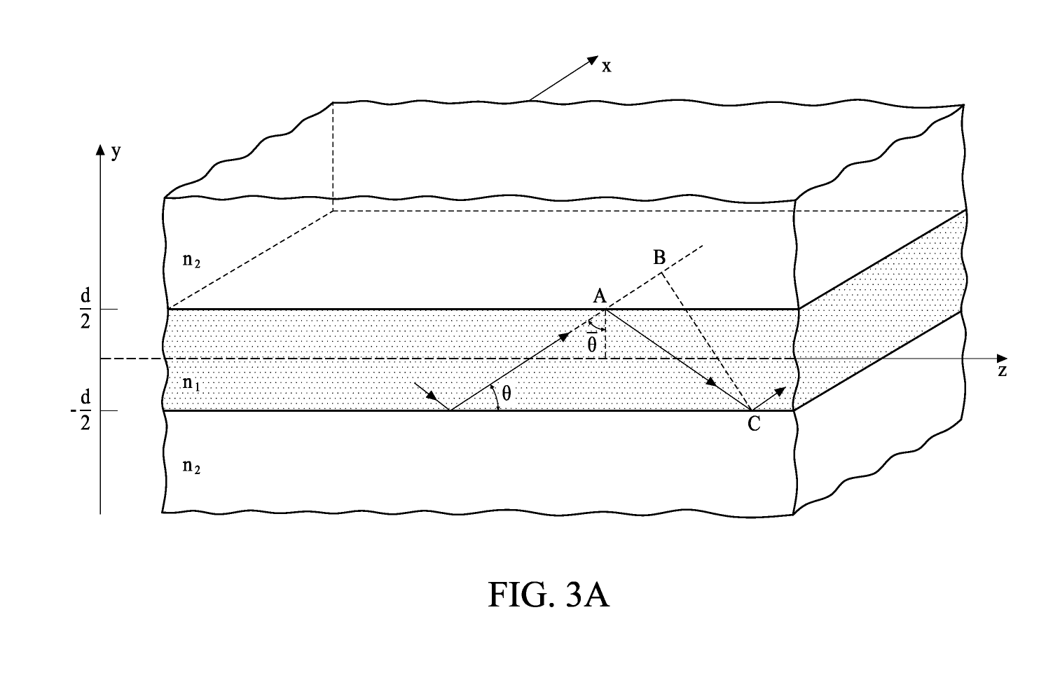

Referring to FIG. 2, the device could also sterilize the surface automatically. For example, if a contaminant 15 such as sweat, grease, dust, bacteria, bacterial strain, microorganism, virus or pathogens, is in contact with or adhered to the front surface 144 of the light guiding member 14 as shown in FIG. 2, some light rays 149 will penetrate the surface (such as the front surface 144) due to the FTIR phenomenon and irradiate the contaminant 15. Therefore, the pathogens in the contaminant 15 are killed by the short wavelength light. Furthermore, any kind of pathogen, like bacteria or virus which adheres on the surface, will be irradiated and sterilized by the evanescent wave, so that the device could provide a germ-free and sterilized surface. FIG. 3A is an illustration of a cross-sectional view showing the formation of guided light rays. As shown in FIG. 3A, the region between +d/2 in y-axis is a dielectric light guiding slab, and the light rays with an angle of less than cos⁻¹(n₂/n₁) are guided inside the slab by total internal reflection. For example, the material of the light guide is α-quartz which has refractive index n₁=1.6 at λ=254 nm, and the region outside ±d/2 in y-axis is air with refractive index n₂=1, so that the light ray with an angle less than cos⁻¹ (n₂/n₁)=51.31° could be guided in the dielectric light guiding slab. On the other hand, the light ray with an angle larger than cos⁻¹(n₂/n₁)=51.31° will pass through the dielectric light guiding slab as shown in FIG. 3B.

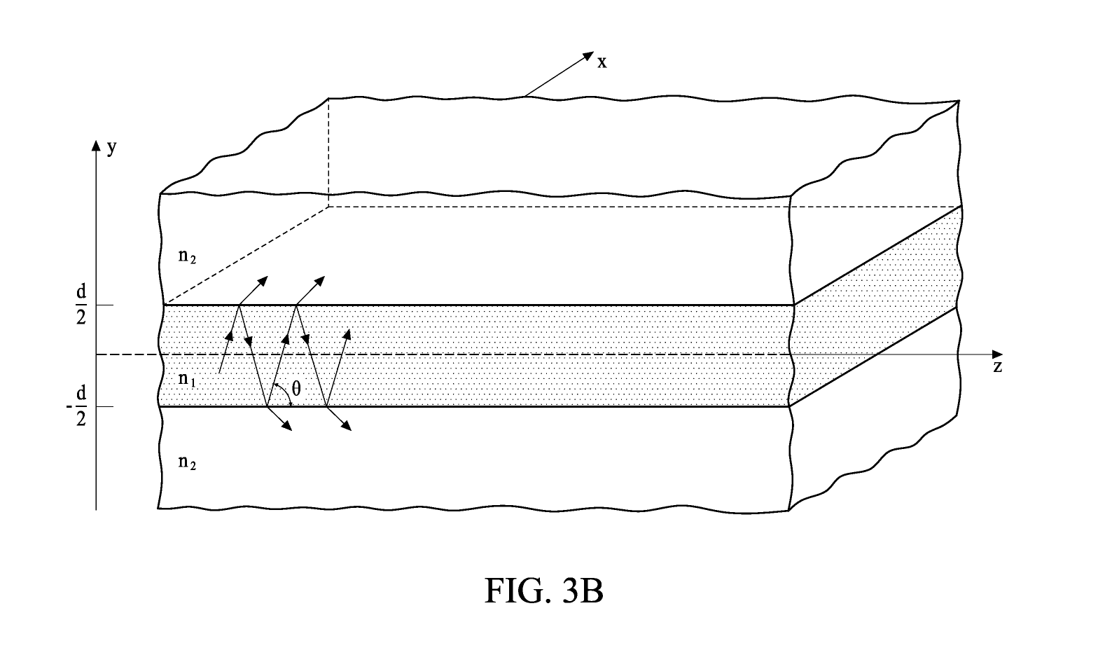

FIG. 4 provides a visual explanation of an evanescent wave. The figure is an example of the field distribution for Transverse-Electric (TE) guided modes in the dielectric light guiding slab. The field outside the slab must match the internal field at the boundary y=±d/2, so that there is an exponentially decaying energy outside the slab. Such well-known energy field outside the slab is said to be an evanescent wave.

As shown in FIG. 1, a user physically touches the front surface 144 of the light guiding member 14 with a finger, wherein the ultraviolet light rays are guided inside the light guiding member 14. Because of the evanescent wave effect, the light rays irradiate the part of the finger which is touching or very close to the front surface 144. Therefore the contact area of the finger and the front surface 144 is disinfected by the ultraviolet light. In addition, the evanescent wave only affects the region within several micrometers outside the surface, so that in applications such as elevator buttons, the ultraviolet light will not irradiate on a user's eyes even if the light source is turned on. Therefore, since the sterilizing device is safe as long as there is a distance of several micrometers between the device and the user, and there is no need to have a shield covering the contact surface of the sterilizing device.

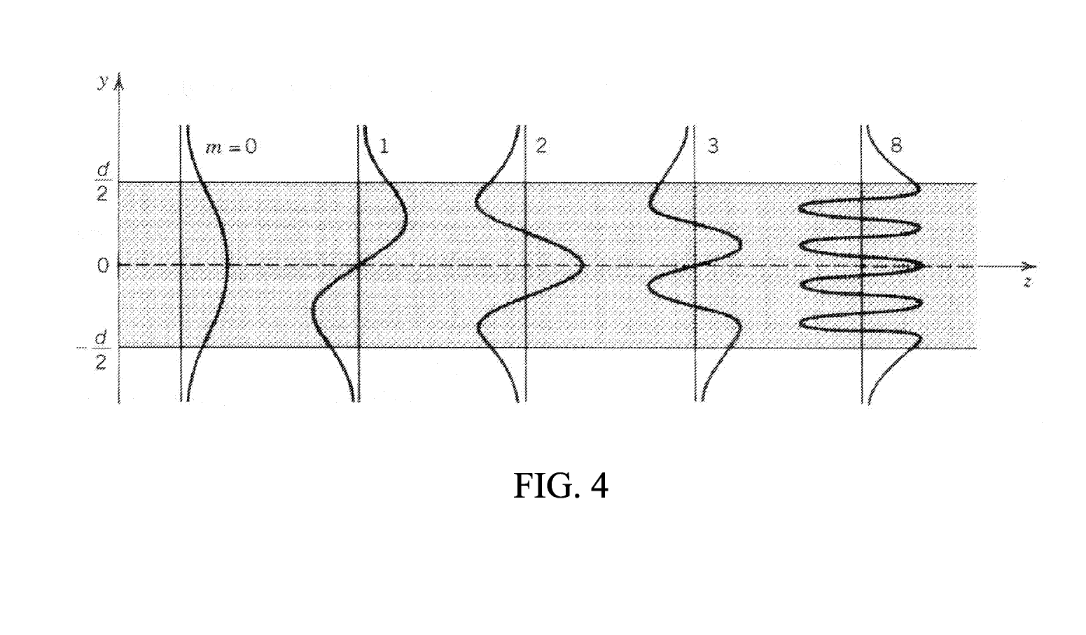

A sterilizing device of the disclosure could be used in a variety of applications, for example, a publicly accessible apparatus having a manual activation device. FIG. 5A shows a cross-sectional view of a sterilizing switch button device 50 in accordance with an exemplary embodiment. The sterilizing switch button device 50 comprises a UV light source 52, a light guiding member 53, a housing 54, a spring 55, and a light guiding member 53. The UV light source 52 is disposed adjacent to a side surface 534 of the light guiding member 53. Therefore, some of the short wave length light rays, radiating from the UV light source 52, are introduced into the light guiding member 53, and then guided within the light guiding member 53. During operation, when the UV light source 52 turns on, any kind of pathogen, like bacteria or virus which adheres to the front surface 532, will be irradiated and sterilized by the short wavelength light rays. In addition, referring to FIG. 5B, when a user touches or presses the button device 50 by his finger, the light rays will irradiate and sterilize the contact area of the finger. When the user touches the button device 50, the spring 55 is compressed so that the light source 52 and the light guiding member 53 move downward and an electrical contact point 56 electrically shorts to the terminals 57. In this embodiment, the sterilizing switch button device 50 is used in an elevator. However, the disclosure should not be limited to the embodiment.

In order to reduce power consumption and increase the life time of UV lamp of the sterilizing switch button device 50, a sensor (not shown) for detecting the touch of the selective buttons could be integrated into the sterilizing switch button device 50. Therefore, the sterilizing switch button device 50 only operates when the user physically touches the selective buttons. Furthermore, a timer (not shown) for setting up the operation time of the sterilizing switch button device 50 could be integrated into the sterilizing device 50. Therefore, the sterilizing switch button device 50 only operates when the timer is activated.

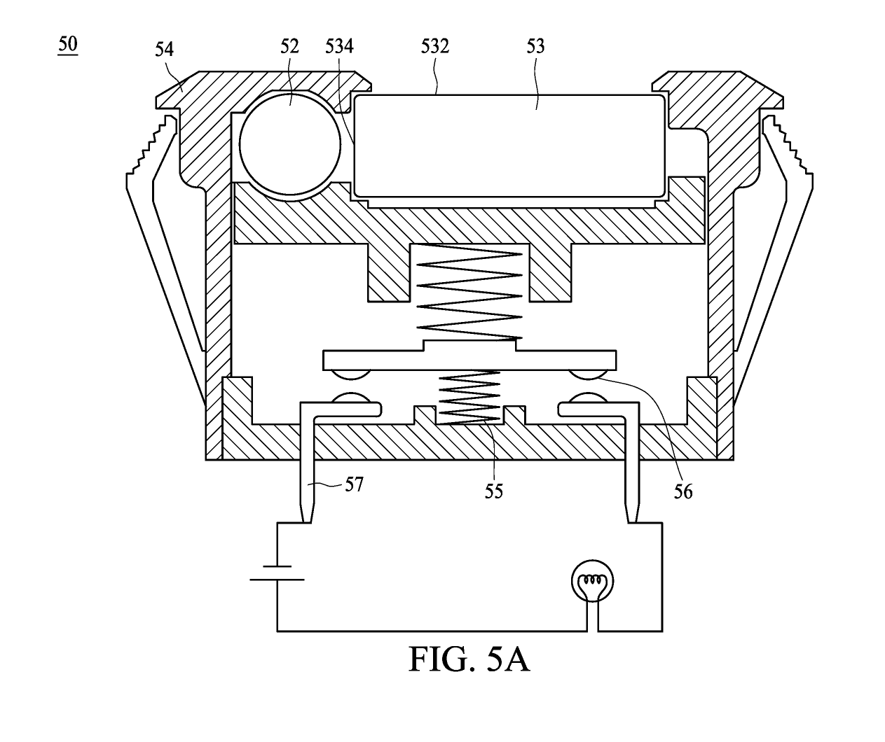

FIG. 6 shows the flow chart of one embodiment of a sterilizing method of the present disclosure. In step 601, the flow starts. In step 602, a sterilizing device determines whether a user is physically touching or closing to the sterilizing device. If YES, a UV light source is turned on in step 603; otherwise, the sterilizing device continues to check for a user touch. In step 603, a timer is also reset or activated according to a predetermined time interval Td. In step 604, if the predetermined time interval Td has passed, then the UV light source is turned off in step 605, and the flow returns to step 602. In one embodiment of the present disclosure, a switch could be used to control the status of the UV light source.

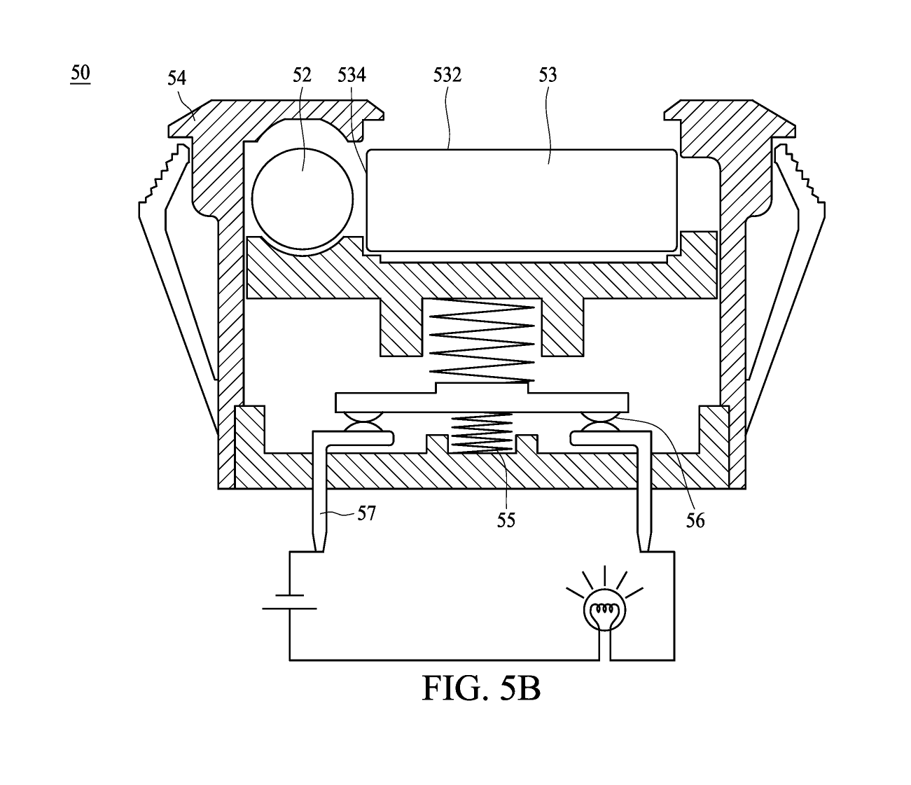

As mentioned before, the device could also sterilize the contact surface when the user's finger not contact. Furthermore, the UV light may cause injury to the skin if there is too much exposure, therefore in order to prevent a user's finger from being irradiated by UV light rays, a UV light source should be turned off upon detection of the touch of the user's finger. FIG. 7 shows the flow chart of another embodiment of a sterilizing method of the present disclosure. In step 701, the flow starts. In step 702, a UV light source is turned on. In step 703, a sterilizing device determines whether a user is physically touching selective buttons. If YES, a timer is turned off in step 704, and then the UV light source is turned off in step 705. In step 706 it is determined whether the timer is activated. In step 707, if the timer is not activated, the timer is reset according to a predetermined time interval Td, and then the timer is turned on in step 708. In step 709, if the timer is activated and a predetermined time interval Td has passed, then the UV light source is turned off in step 705; otherwise, the flow returns to step 702. In one embodiment of the present disclosure, a switch could be used to control the status of the UV light source.

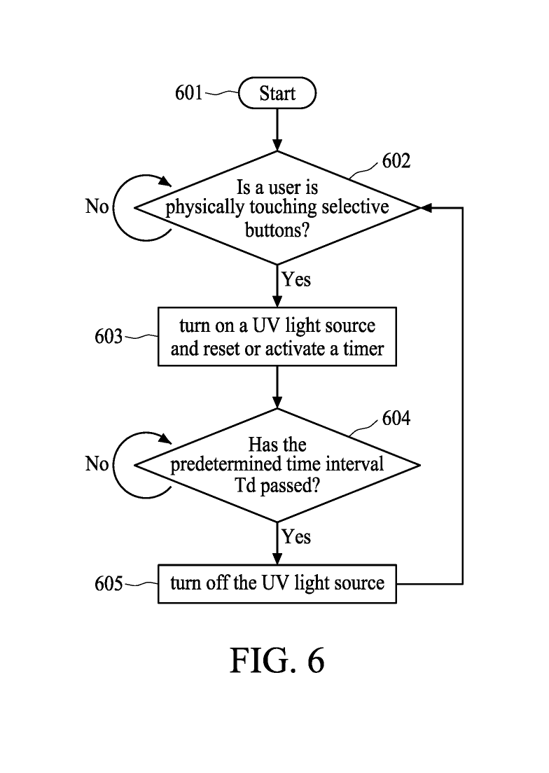

According to another embodiment, a sterilizing device could be implemented as a touch panel. FIG. 8 shows a cross-sectional view of the sterilizing touch panel 60 in accordance with an exemplary embodiment. The sterilizing device 60 comprises a UV light source 61, a light guiding member 62, a spacer 63, a transparent touch screen 64, and a display layer 65. Referring to FIG. 8, the transparent touch screen 64 is formed on the display layer 65, and the spacer 63 is disposed between the transparent touch screen 64 and the light guiding member 62. In addition, a flex circuit 66 is electrically coupled between the transparent touch screen 64 and an integrated circuit chip 67. In one embodiment of the present disclosure, the transparent touch screen 64 is a projected capacitive touch screen comprising a grid pattern of multiple vertical transparent electrodes that cross multiple horizontal electrodes. The display layer 65 could be, for example, an In Plane Switching (IPS) liquid crystal display panel, a Twisted Nematic (TN) liquid crystal display panel, a Vertical Alignment (VA) liquid crystal display panel, or an Organic Light-Emitting Diode (OLED) display panel.

In another embodiment of the present disclosure, the spacer 63 could be a transparent layer, and the refractive index of the transparent layer is lower than or the same as that of the light guiding member 62. For example, the light guiding member 62 could be made from fused silica (the refractive index n=1.51 @ 250 nm), and the spacer 63, which coats on the light guiding member 62, could be made from CaF₂ (the refractive index n=1.47 @ 250 nm).

Referring to FIG. 8, the light guiding member 62 is made of a transparent material, such as glass or quartz, and has side surfaces 622 and a front surface 624. The light source 61 is disposed adjacent to the side surface 622 of the light guiding member 62. During operation, when a user physically touches the front surface 644 of the light guiding member 62, some UV light rays pass out of the light guiding member 62 due to the FTIR phenomenon, so that the user's finger and the contact area could both be disinfected. However, any kind of pathogen, like bacteria or virus which adheres to the front surface 644, will be irradiated and sterilized by the UV light rays cause by the FTIR phenomenon, so that the front surface 644 could be a germ-free and sterilized surface.

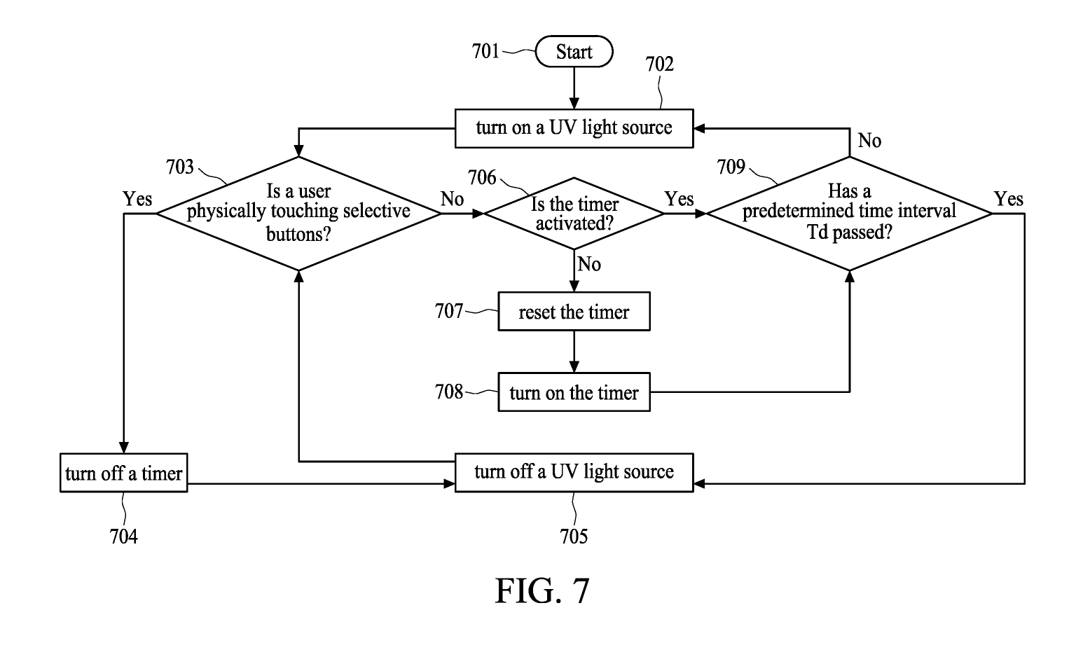

According to yet another embodiment, a sterilizing device could be implemented as a door handle. FIG. 9A shows a sterilizing device 70 in accordance with an exemplary embodiment. The sterilizing device 70 comprises a UV light source 74, a handle 71, connection portions 73, and seal caps 72. As shown in FIG. 9A, the UV light source 74 is disposed between the seal cap 72 and the handle 71. The handle 71 has a cylinder shape and is made of UV penetrating material, such as quartz or fused silica. The handle 71 acts as a light guiding member. Referring to FIG. 9A, the connection portions 73 are attached to the seal cap 72 so that a user could open or close the door by the connection portions 73.

FIG. 9B shows one embodiment of the sterilizing device 70 of FIG. 9A with more detail. Referring to FIG. 9B, the handle 71 has a solid cylinder shape, and a collimating lens 75 is disposed between the handle 71 and the UV light source 74. The light rays from the light source 74 are collimated through the collimating lens 75 and then enter a front surface 711 of the handle 71, and then the UV light rays are guided in the handle 71. During operation, when a user physically touches the outer surface 712 of the handle 71, an evanescent wave goes out of the surface 712 of the handle 71 and irradiate at the contact area of the skin. Furthermore, any kind of pathogen, like bacteria or virus which adheres on the outer surface 712, will be irradiated and sterilized by the evanescent wave, so that the outer surface 712 of the door handle 71 could be a germ-free and sterilized surface.

FIG. 9C shows another embodiment of the sterilizing device 70 of FIG. 9A with more detail. Referring to FIG. 9C, the handle 71 has a hollow cylinder shape, and two collimating lens 75′ are disposed between the handle 71 and the UV light sources 74′. The light rays from the light source 74′ are collimated through the collimating lens 75′ and then enter a front surface 711 of the handle 71. Therefore, when an object contacts or comes close to the surface 712 of the handle 71, an evanescent wave from the UV light rays irradiates on the object.

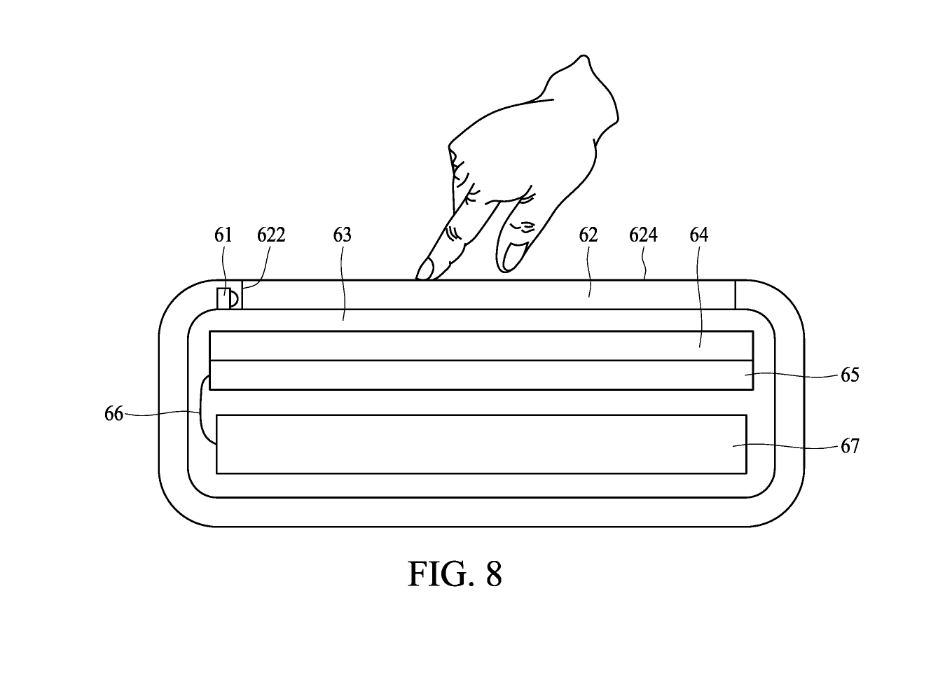

The UV light source in the aforementioned embodiments is disposed adjacent to the side surface of the light guiding member. However, the present disclosure should not be limited to the embodiments. FIG. 10 shows another arrangement of a UV light source in a sterilizing device in accordance with an exemplary embodiment. Referring to FIG. 10, a prism 102 is formed on a peripheral surface 1044 of a rear surface 1042 of a light guiding member 104, and the position of a light source 106 is slightly different from that of FIG. 1. The light source 106 is disposed at a position relative to the light guiding member 104 such that the light rays from the light source 106 enter the rear surface 1042 of the light guiding member 104 from the peripheral surface 1044 of the light guiding member 104 through the prism 102, and then are repeatedly reflected totally within the light guiding member 104.

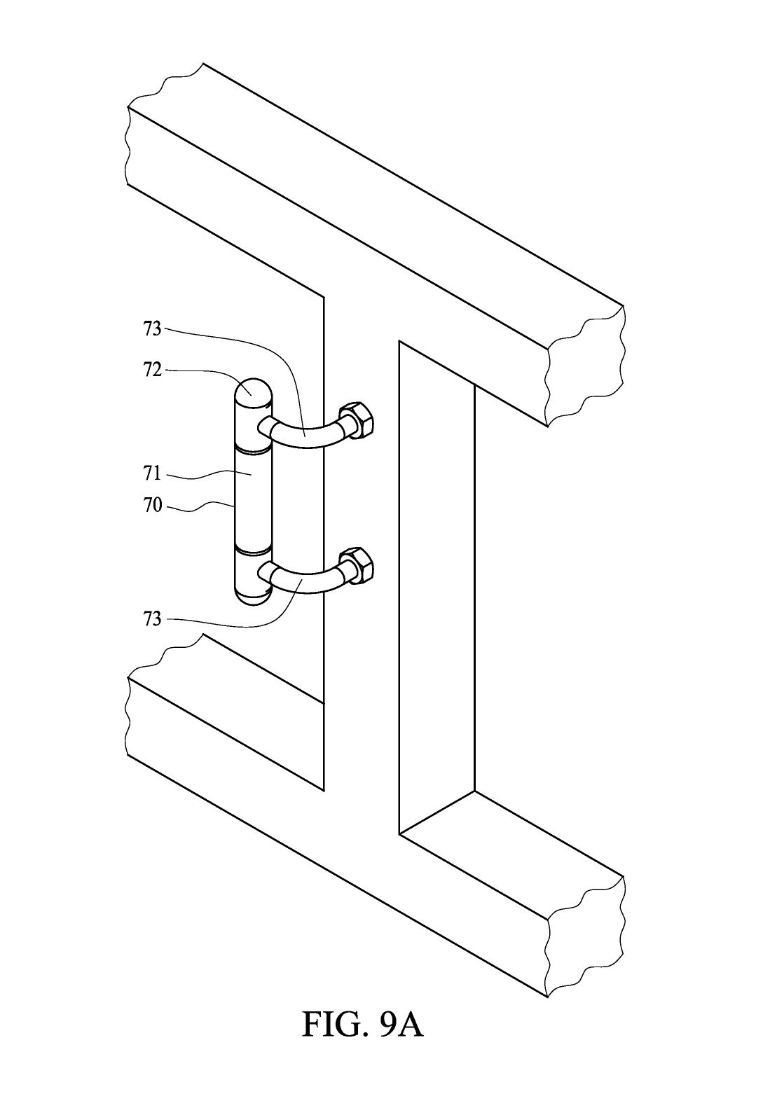

FIG. 11 shows another arrangement of a UV light source in a sterilizing device in accordance with an exemplary embodiment. Referring to FIG. 11, a tapered peripheral surface 1047 is formed adjacent to a front surface 1046′ of the light guiding member 104′. An optic fiber 108 is directed towards the peripheral surface 1047 and is used to couple the light rays from a light source. The light rays enter the light guiding member 104′ from the peripheral surface 1047 and then are repeatedly reflected totally within the light guiding member 104′.

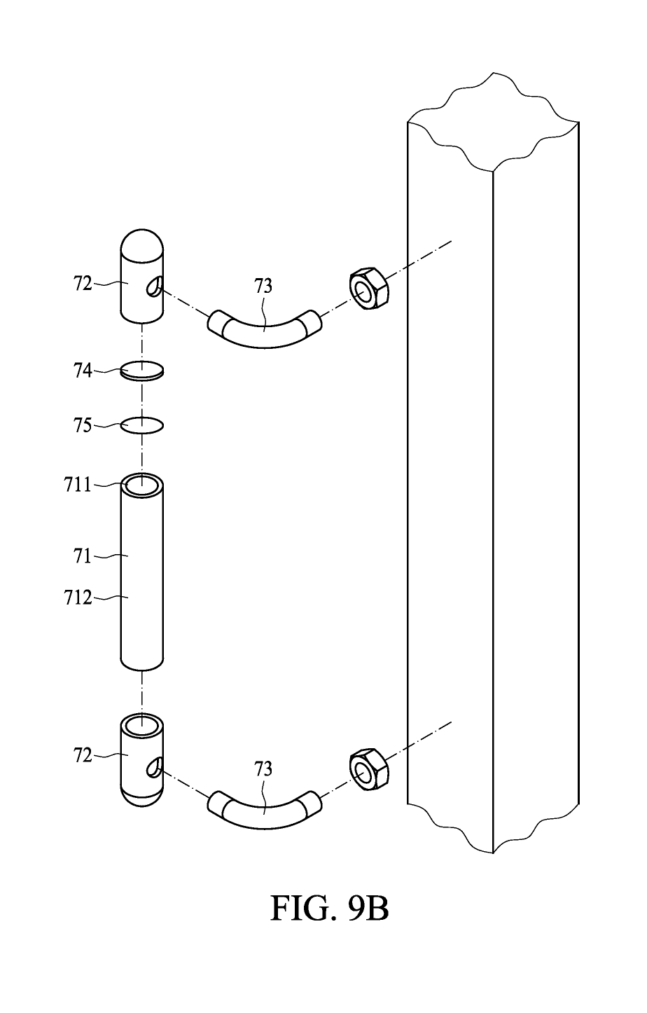

FIG. 12 shows another arrangement of a UV light source in a sterilizing device in accordance with an exemplary embodiment. Referring to FIG. 12, a tapered peripheral surface 1047″ is formed adjacent to a rear surface 1042″ of the light guiding member 104″. A hologram (not shown) could be formed on the tapered peripheral surface 1047″ to enhance the efficiency of the light introduced to the light guiding member 104″. An optic fiber 108″ is directed towards the peripheral surface 1047″ and is used to couple the light rays from a light source. The light rays enter the light guiding member 104″ from the peripheral surface 1047″ and then are repeatedly reflected totally within the light guiding member 104″.

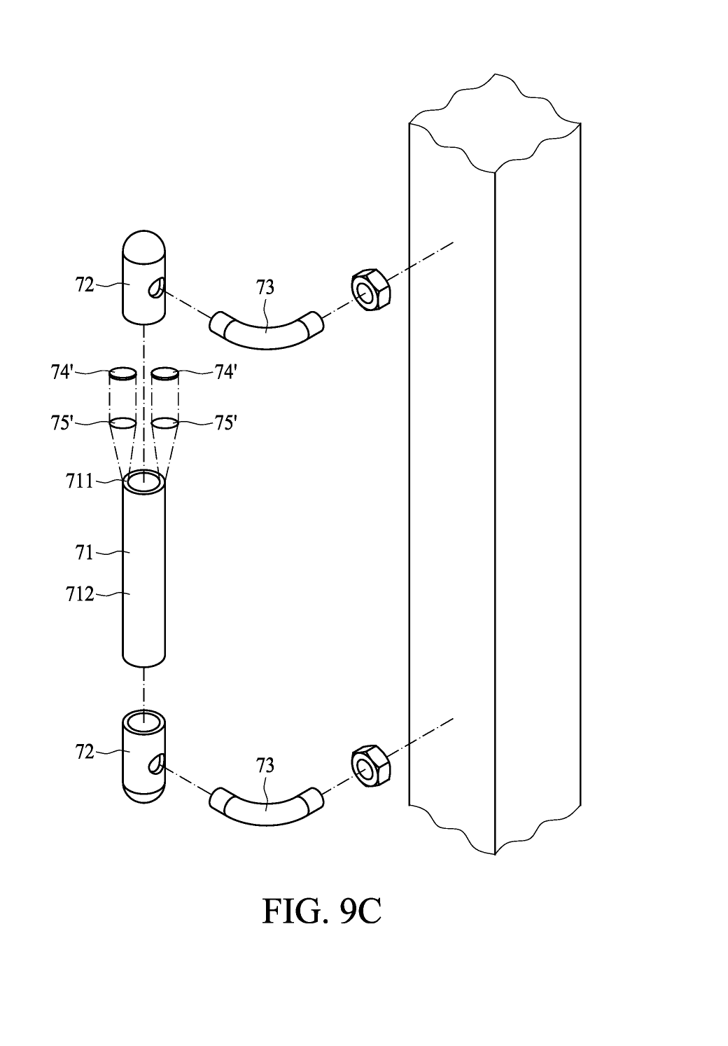

The UV light source shown in the aforementioned embodiments is disposed adjacent to the side surface of the light guiding member. However, the present disclosure should not be limited to the embodiments. FIG. 13 shows another arrangement of a UV light source in a sterilizing device in accordance with an exemplary embodiment. Referring to FIG. 13, a collimating lens 114 and a prism 116 are disposed on a front surface 1182 of a light guiding member 118. The light rays from a light source 112 are collimated through the collimating lens 114 and are incident on the prism 116. Next, the light rays incident on the prism 116 enter the front surface 1182 of the light guiding member 118 and are repeatedly reflected totally within the light guiding member 118.

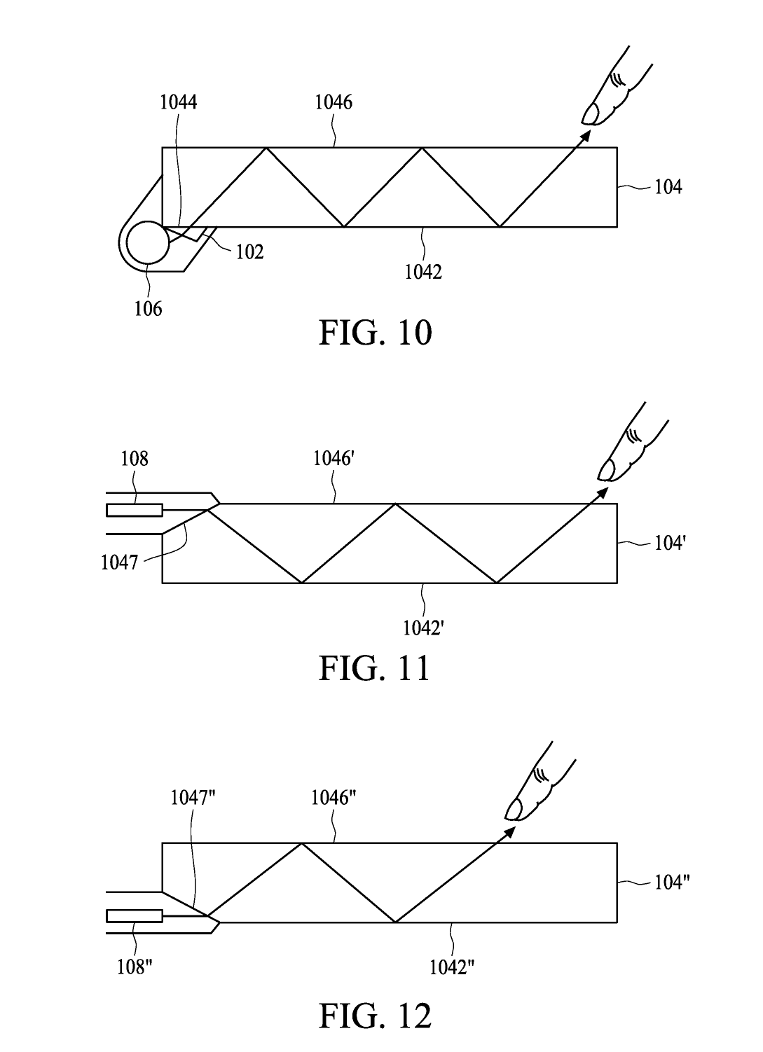

FIG. 14 shows another arrangement of a UV light source in a sterilizing device in accordance with an exemplary embodiment. Referring to FIG. 14, a grating 115 is formed on an external front surface 1182′ of a light guiding member 118′. When the light rays emitted from a UV light source 112′ are incident on the light guiding member 118′, the incident light rays are diffracted by the grating 115 and then are totally reflected within the light guiding member 118′. The grating 115 could be replaced with a hologram, wherein the grating is an optical component with a constant periodic structure while the hologram is an optical component with a varied periodic structure.

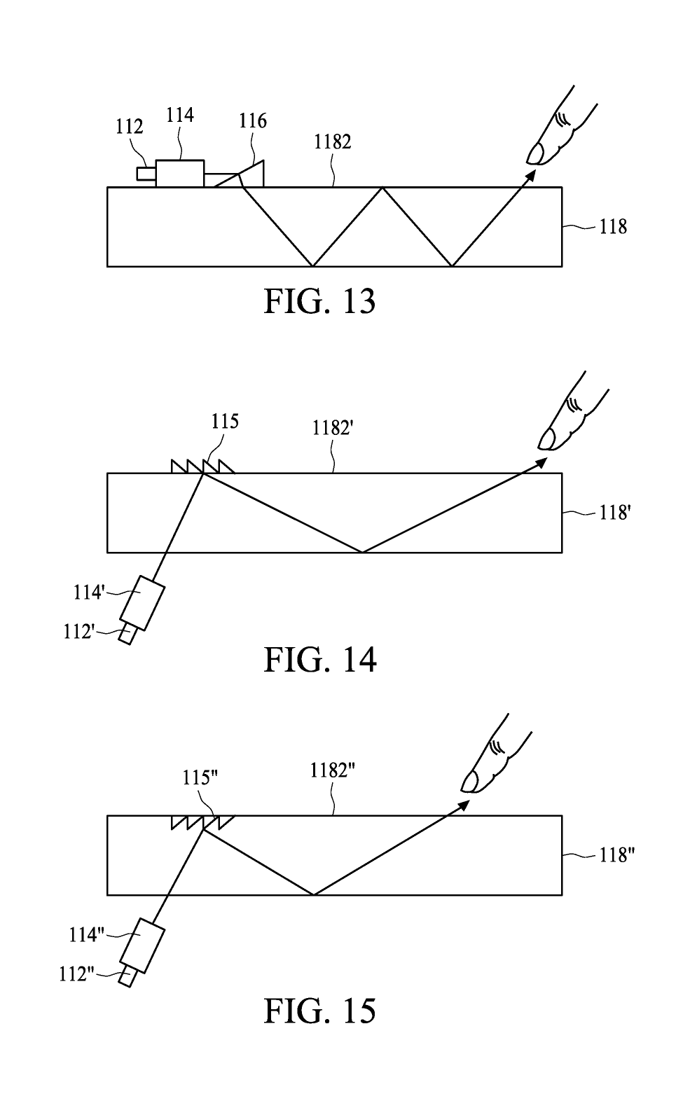

In addition, a grating 115″ could be formed inside on an internal front surface 1182″ of a light guiding member 118″ as shown in FIG. 15. Therefore, the light rays from the collimating lens 114″ are diffracted by the grating 115″ and then are totally reflected within the light guiding member 118″.

The scope of the present application is not intended to be limited to the particular embodiments of the process, machine, manufacture, composition of matter, means, methods and steps described in the specification. As one of ordinary skill in the art will readily appreciate from the disclosure of the disclosure, processes, machines, manufacture, compositions of matter, means, methods, or steps, presently existing or later to be developed, that perform substantially the same function or achieve substantially the same result as the corresponding embodiments described herein may be utilized according to the disclosure. Accordingly, the appended claims are intended to include within their scope such processes, machines, manufacture, compositions of matter, means, methods, or steps.

What is claimed is:

1. A sterilizing device, comprising:
a light guiding member having a tapered peripheral surface and a front surface connected to the tapered peripheral surface, wherein the tapered peripheral surface is formed adjacent to the light guiding member, the light guiding member is an unitary element, and a material of the light guiding member is free of metal; and
an ultraviolet (UV) light source for emitting UV light rays so that the UV light rays are guided into the light guiding member from the tapered peripheral surface and propagate inside the light guiding member based on a total internal reflection,
wherein when an object contacts or comes close to the front surface, the UV light rays emit out from the front surface of the light guiding member and irradiate on the object due to a frustrated total internal reflection phenomenon.

2. The sterilizing device of claim 1, wherein the object comprises a microorganism, and when the object leaves the front surface, the UV light rays are totally reflected internally inside the light guiding member and does not transmit energy across the front surface.

3. The sterilizing device of claim 1, wherein the object comprises mammalian epidermis, and when the object leaves the front surface, the UV light rays are totally reflected internally inside the light guiding member and does not transmit energy across the front surface.

4. The sterilizing device of claim 1, wherein the light guiding member has a smooth area on the front surface.

5. The sterilizing device of claim 1, wherein the light guiding member has a solid cylinder shape or a hollow cylinder shape.

6. The sterilizing device of claim 1, wherein a collimating lens is disposed between the light guiding member and the UV light source.

7. The sterilizing device of claim 1, further comprising a sensor configured to sense the object contacts or comes close to the front surface.

8. The sterilizing device of claim 1, further comprising:
a switch configured to control a status of the UV light source; and
a timer configured to control the switch according to a predetermined time interval.

9. The sterilizing device of claim 1, wherein a prism, a grating or a hologram is disposed between the light guiding member and the UV light source.

10. The sterilizing device of claim 1, wherein the light guiding member is made of the material selected from the group consisting of glass, borosilicate glass, fused silica, quartz, sapphire, plastic, resin, and polymers.

11. The sterilizing device of claim 1, wherein the light guiding member is flexible.

12. The sterilizing device of claim 1, wherein the light guiding member further has a rear surface opposite to front surface, the sterilizing device further comprises a first covering member covering the tapered peripheral surface and a part of the front surface and the rear surface adjacent to the UV light source, and the first covering member is not UV light transmissive.

13. The sterilizing device of claim 1, wherein the light guiding member further has a first side surface, a second side surface, and a rear surface, the front surface and the rear surface are opposite to each other, and the first side surface and the second side surface are opposite to each other, the sterilizing device further comprises a second covering member covering the second side surface and a part of the front surface and the rear surface, and the second covering member is not UV light transmissive.

14. A sterilizing touch panel, comprising:
a display layer;
a transparent touch screen formed on the display layer;
a light guiding member having a tapered peripheral surface and a front surface connected to the tapered peripheral surface, wherein the tapered peripheral surface is formed adjacent to the light guiding member, the light guiding member is an unitary element, and a material of the light guiding member is free of metal;
a spacer disposed between the transparent touch screen and the light guiding member; and
an ultraviolet (UV) light source for emitting UV light rays so that the UV light rays are guided into the light guiding member from the tapered peripheral surface and propagate inside the light guiding member based on a total internal reflection,
wherein when an object contacts or comes close to the front surface, the UV light rays emit out from the front surface of the light guiding member and irradiate on the object due to a frustrated total internal reflection phenomenon.

15. The sterilizing touch panel of claim 14, wherein the object comprises a microorganism, and when the object leaves the front surface, the UV light rays are totally reflected internally inside the light guiding member and does not transmit energy across the front surface.

16. The sterilizing touch panel of claim 14, wherein the object comprises mammalian epidermis, and when the object leaves the front surface, the UV light rays are totally reflected internally inside the light guiding member and does not transmit energy across the front surface.

17. The sterilizing touch panel of claim 14, wherein the light guiding member has a smooth area on the front surface.

18. The sterilizing touch panel of claim 14, wherein a collimating lens is disposed between the light guiding member and the UV light source.

19. The sterilizing touch panel of claim 14, wherein a prism, a grating or a hologram is disposed between the light guiding member and the UV light source.

20. The sterilizing touch panel of claim 14, wherein the light guiding member is made of the material selected from the group consisting of glass, borosilicate glass, fused silica, quartz, sapphire, plastic, resin, and polymers.

21. The sterilizing touch panel of claim 14, wherein the spacer is a transparent layer, and a refractive index of the transparent layer is lower than or the same as the refractive index of the light guiding member.

22. The sterilizing touch panel of claim 14, wherein the light guiding member further has a rear surface opposite to front surface, the sterilizing touch panel further comprises a first covering member covering the tapered peripheral surface and a part of the front surface and the rear surface adjacent to the UV light source, and the first covering member is not UV light transmissive.

23. The sterilizing touch panel of claim 14, wherein the light guiding member further has a first side surface, a second side surface, and a rear surface, the front surface and the rear surface are opposite to each other, and the first side surface and the second side surface are opposite to each other, the sterilizing touch panel further comprises a second covering member covering the second side surface and a part of the front surface and the rear surface, and the second covering member is not UV light transmissive.

24. A manufacturing method for a sterilizing device, comprising:
providing a light guiding member having a tapered peripheral surface and a front surface connected to the tapered peripheral surface, wherein the tapered peripheral surface is formed adjacent to the light guiding member, and the light guiding member is an unitary element;
providing an ultraviolet (UV) light source that emits UV light rays so that the UV light rays are guided into the light guiding member from the tapered peripheral surface and propagate inside the light guiding member based on a total internal reflection, wherein a material of the light guiding member is free of metal; and
providing an object, wherein when the object contacts or comes close to the front surface, the UV light rays emit out from the front surface of the light guiding member and irradiate on the object due to a frustrated total internal reflection phenomenon.

25. The manufacturing method of claim 24, further comprising:
totally reflecting the UV light beam internally inside the light guiding member and keeping the energy of the UV light beam in the light guiding member which the object leaves the front surface, wherein the object comprises a microorganism.

26. The manufacturing method of claim 24, further comprising:
totally reflecting the UV light beam internally inside the light guiding member and keeping the energy of the UV light beam in the light guiding member when the object leave the front surface, wherein the object comprises mammalian epidermis.

27. The manufacturing method of claim 24, further comprising:
totally reflecting the UV light beam internally inside the light guiding member and keeping the energy of the UV light beam in the light guiding member when the object leave the front surface, wherein the light guiding member has a smooth area on the front surface.

28. The manufacturing method of claim 24, further comprising:
totally reflecting the UV light beam internally inside the light guiding member and keeping the energy of the UV light beam in the light guiding member when the object leave the front surface, wherein the light guiding member has a solid cylinder shape or a hollow cylinder shape.

29. The manufacturing method of claim 24, further comprising:
totally reflecting the UV light beam internally inside the light guiding member and keeping the energy of the UV light beam in the light guiding member when the object leave the front surface, wherein a collimating lens is disposed between the light guiding member and the UV light source.

30. The manufacturing method of claim 24, further comprising:
totally reflecting the UV light beam internally inside the light guiding member and keeping the energy of the UV light beam in the light guiding member when the object leave the front surface; and
sensing with a sensor when the object contacts or comes close to the front surface.

31. The manufacturing method of claim 24, further comprising:
totally reflecting the UV light beam internally inside the light guiding member and keeping the energy of the UV light beam in the light guiding member when the object leave the front surface, wherein the sterilizing device further comprising:
a switch is configured to control the status of the UV light source; and
a timer is configured to control the switch according to a predetermined time interval.

32. The manufacturing method of claim 24, further comprising:
totally reflecting the UV light beam internally inside the light guiding member and keeping the energy of the UV light beam in the light guiding member when the object leave the front surface, wherein a prism, a grating or a hologram is disposed between the light guiding member and the UV light source.

33. The manufacturing method of claim 24, further comprising:
totally reflecting the UV light beam internally inside the light guiding member and keeping the energy of the UV light beam in the light guiding member when the object leave the front surface, wherein the light guiding member is made of the material selected from the group consisting of glass, borosilicate glass, fused silica, quartz, sapphire, plastic, resin, and polymers.

34. The manufacturing method device of claim 24, further comprising:
totally reflecting the UV light beam internally inside the light guiding member and keeping the energy of the UV light beam in the light guiding member when the object leave the front surface, wherein the light guiding member is flexible.
